(19) United States
(12) Patent Application Publication
(10) Pub. No.: US 2021/0186352 A1
(43) Pub. Date: Jun. 24, 2021

(54) Operating an MR System and an MR System

(71) Applicant: Siemens Healthcare GmbH, Erlangen (DE)

(72) Inventor: Stephan Biber, Erlangen (DE)

(21) Appl. No.: 17/120,490

(22) Filed: Dec. 14, 2020

(30) Foreign Application Priority Data
Dec. 20, 2019 (EP) 19218683.1

(51) Int. Cl. A61B 5/055; A61B 5/01; A61B 5/0507; G01R 33/28

(52) U.S. Cl. CPC A61B 5/055; G01R 33/288; A61B 5/0507; A61B 5/015

(57) ABSTRACT

A method is used to operate an MR system having at least one MR body coil and a control device connected to the at least one MR body coil. At least one radiometer is used to measure a body temperature of a body region, which body region can be illuminated by the respective radiometer, of a patient to be examined by the MR system. The measured body temperature is compared with a limit temperature, and an MR transmit power directed at the patient is brought closer to the limit temperature on the basis of a result of the comparison. The radiometer operates, in particular, in a frequency band that differs from the MR band. An MR body coil for performing the method has at least one radiometer antenna and an amplifier connected downstream of the radiometer antenna. The MR body coil can also have an input blocking filter connected downstream of the radiometer antenna and designed to block the MR band.

### RELATED CASE

This application claims the benefit of European Application EP19218683.1, filed on Dec. 20, 2019, which is hereby incorporated by reference in its entirety.

### FIELD

The present embodiments relate to a method for operating an MR system comprising at least one magnetic resonance (MR) body coil and a control device that is connected to the at least one MR body coil, wherein at least one radiometer is used to measure the body temperature of a body region of a patient who is to be examined by the MR system. The body region can be illuminated by the respective radiometer, The present embodiments further relate to an MR body coil that is configured to perform the method and an MR system that is configured to perform the method.

### BACKGROUND

In the field of nuclear spin tomography (MRT), MR transmission systems are used to excite the spins in the body of a patient. Medium powers in the range of several 100 W are emitted for this purpose, though very high peak powers in the range of 1 kW to 40 kW are also used. This presents a problem in that, particularly when using high-field systems with a Bo field strength of more than one Tesla, medium powers, measured with specific absorption rate (SAR) in W/kg, absorbed in the patient are so high that the powers must be monitored in order to comply with permitted SAR limit values. MR body models are often stored for the purpose of determining the SAR limit values, and a position of the patient is estimated. On the basis of this information, the requirements of the MR sequence(s) of an MR scan, and reflection factors of the MR transmit coil(s), it is possible to estimate how much medium MR transmit power can be emitted without exceeding the SAR limit values. In order that the safety of the patient can be guaranteed under all conditions, high safety factors are included, disadvantageously resulting in greater restrictions in the MR transmit power than is necessary in many cases.

AbdEl-Monem M. El-Sharkawy et al., “Absolute Temperature Monitoring Using RF Radiometry in the MRI Scanner”, IEEE Trans Circuits Syst I Regul Pap. Author manuscript; available in PMC 2007 Apr. 10, Published in final edited form as: IEEE Trans Circuits Syst I Regul Pap. 2006 November; 53(11): 2396-2404, relate to temperature capture by microwave radiometry for non-invasive measurement of an absolute temperature of tissues in a body. Such clinical radiometers work in the gigahertz range, however, their penetration depth is limited. A non-invasive radiometer is therefore proposed that works at low radio frequencies (64 MHz) with a bandwidth of 100 kHz using an external HF loop coil as a thermal detector. The radiometer uses a precise impedance measurement and an automatic adaptation circuit with an accuracy of 0.05Ω in order to balance out any load fluctuations. The radiometer allows temperature measurements with an accuracy of ±0.1° K over a tested physiological range of 28° C. to 40° C. in salt phantoms whose electrical properties correspond to those of tissue. Since 1.5-T magnetic resonance tomography systems (MRT) also work at 64 MHz, it is clear that a radiometer can be integrated into an MRT scanner for the purpose of monitoring an HF power output and for temperature dosimetry, in order to obtain approximate locally resolved absolute heat maps in the physiological range. It is deduced from this that HF radiometry is very promising as a direct non-invasive method for monitoring tissue heating during MRT measurements and therefore represents an independent check of patient safety.

### SUMMARY AND DETAILED DESCRIPTION

The object is to overcome at least partially the disadvantages of the prior art and in particular to provide an improved way of adapting MR transmit (TX) power during an MR examination of a patient.

A method is provided for operating an MR system having at least one MR body coil and a control device that is connected to the at least one MR body coil. At least one radiometer is used to measure the body temperature of a body region, which body region can be illuminated by the respective radiometer, of a patient who is to be examined by the MR system. The measured body temperature is compared with a limit temperature. An MR transmit power directed at the patient is brought closer to the limit temperature on the basis of a result of the comparison, but in particular is not exceeded. In other words, an MR transmit power directed at the patient is adjusted on the basis of a result of the comparison, such that the body temperature, which can be measured in particular by the at least one radiometer, is brought closer to the limit temperature, but is not exceeded.

This method has the advantage that at least one body temperature can be determined quickly and non-invasively during an MR measuring procedure (also known as an “MR scan”). This in turn can be used to adapt the MR transmit power (also known as “MR TX power”) directed at the patient to prescribed limit body temperatures. It is thereby possible to ensure that a permitted MR transmit power is fully utilized, so that an image quality and/or measuring speed can be increased. It is further advantageous that, e.g. in contrast with IR imaging, the body temperature can also be established or measured in the interior of the patient, which allows even closer approximation to limit body temperatures. In this case, it is also possible to measure spatial temperature distributions in the patient such that the MR transmit power can also be adapted as a function of local temperatures, thereby allowing even more reliable compliance with limit values and/or even better utilization of the MR transmit power.

The MR system includes at least one MR body coil and a control device connected to the at least one MR body coil. The MR body coil includes at least one or more MR receive antennas, which receive a signal response of the patient to MR signals (also known as “MR TX signals”, e.g. MR pulses) that are directed at the patient, and which transfer said signal response to the MR system. The MR TX signals can be generated, e.g. by an MR device (also known as “MR scanner”) and/or by MR transmit antennas on the body coil. In the latter case, the MR transmit antennas can correspond to the MR receive antennas or different antennas. Such an MR system (excluding the radiometric temperature measurement) is generally known and is therefore not explained further.

A body region that can be illuminated by a radiometer is understood to be in particular a body region whose temperature can be determined or measured by said radiometer. In this case, the body temperature for this body region can be determined as a value in particular. A temperature distribution of the patient can be measured, e.g., by an arrangement of a plurality of radiometers and different body regions that can be illuminated, also known as “spatial diversity.” The principle of radiometric measurement of a body temperature is assumed to be generally known.

According to a development, the body temperature of the patient to be examined is measured at different depths of the patient by the at least one radiometer. It is then advantageously possible also to adapt the MR transmit power as a function of temperatures at locally differing depths, thereby allowing even safer compliance with limit values and/or even better utilization of the MR transmit power. The measurement of the body temperature at different depths can be realized, e.g., by evaluating the radiometer signal picked up by the at least one radiometer in different frequencies and/or frequency ranges (e.g. at 500 MHz, 1 GHz, 2 GHz, 5 GHz and/or 10 GHz), since anatomies located at different depths in the body can be examined using different frequencies. This is also known as “frequency diversity.” In order to determine local body temperatures in three dimensions (e.g. in the plane through the plurality of radiometers and in depth), it is particularly advantageous to arrange a plurality of radiometers at different spatial positions with different illumination regions, and to evaluate their radiometer signals in various frequencies and/or frequency ranges.

The radiometer antenna typically used for the radiometric temperature measurement is in particular a different antenna from an MR antenna. This has the advantage that any interference in the radiometric temperature measurement due to MR signals can be kept down. It is then also possible for a bandwidth of the frequency band or useful band for the radiometric temperature measurement to be kept particularly wide, whereas MR antennas disadvantageously tend to be configured in a narrow band on the MR frequencies.

The limit temperature is, in particular, a specified maximum limit temperature. This can be specified, e.g., in order to prevent harm to the patient. According to a development, precisely one limit temperature is specified for a patient. According to a development, individual and possibly different limit temperatures are specified for different body regions.

The limit temperature(s) can be obtained on the basis of, e.g., physiological considerations. For example, the limit temperature(s) can be derived from the SAR (“specific absorption rate”) that a patient should not exceed during an MR scan. Comparing the measured body temperature with a limit temperature can however also include converting the measured body temperature into SAR values and comparing these converted SAR values with SAR limit values.

That an MR transmit power directed at the patient is brought closer to the limit temperature on the basis of a result of the comparison, but is not exceeded, can mean for example that: if the measured body temperature lies below the (possibly local) limit temperature, the MR transmit power is increased until the limit temperature is reached or is about to be reached; or if the measured body temperature lies above the (possibly local) limit temperature, the MR transmit power is decreased until the limit temperature is reached again or is somewhat lower.

The method can be performed automatically by the MR device. Alternatively or additionally, the MR device, in particular the control device thereof, can be configured to adjust the MR transmit power following corresponding input from a user.

The method can use one or more radiometers. A plurality of radiometers can be used, in particular, to measure the temperature at different body regions and thus to measure a temperature distribution of a patient. That the measured body temperature is compared with a limit temperature can therefore include, e.g., cases as follows: body temperatures measured by different radiometers are compared with a limit temperature which is identical for all body regions; or body temperatures measured by different radiometers are compared with individual limit temperatures for the body regions.

According to an embodiment, the radiometer is a Dicke radiometer that includes an antenna (“radiometer antenna”), a noise source and a Dicke switch. The Dicke switch is switched over between the radiometer antenna and the noise source alternately with a specific frequency. A Dicke radiometer advantageously allows a particularly simple structure. As a result of said switching over, thermal drift effects that are significantly longer than the switchover time of the Dicke switch are eliminated (Dicke principle), wherein a typical frequency for the switchover lies in the range between 5 Hz and 50 kHz.

The measured signal picked up by the radiometer antenna is a noise signal corresponding to a thermal (Planckian) noise of the patient. The noise source (also known as “noise generator” or “reference noise source”) generates a reference noise signal. Unless otherwise suggested by the context, a noise signal can therefore be understood in the following to mean both the reference noise signal that is artificially generated by the noise source and the measured signal that is picked up by the radiometer antenna. The noise source can be, for example, a 50-Ohm resistance or a calibrated noise source based on a noise diode, for example.

By comparing the two noise powers of radiometer antenna (measured signal) and noise source (reference noise signal), it is possible to determine the noise power of the measured signal. On the basis of the knowledge of the noise power P of the measured signal received by the radiometer antenna, the bandwidth B of the useful band, and the chain gain G of the receive chain, the temperature T measured by the radiometer antenna can be determined absolutely by T=P/(k·B·G), where k is Boltzmann's constant. The structure and operation of Dicke radiometers are generally known and are therefore not explained further here.

According to an embodiment, the Dicke switch is connected to the noise source during transmit phases of the MR system, and to the radiometer antenna during non-transmit phases of the MR system. This has the advantage that any interference in the temperature measurement via the radiometer antenna due to the MR transmit signals is specifically prevented, and possible measuring times can nonetheless be used particularly effectively. In other words, the radiometer uses the time of the transmit phases to pick up the noise signal from the noise source serving as a reference, while in the non-transmit phases the radiometer is switched to the object or the patient and in this way picks up the measured signal from the radiometer antenna. A transmit phase is understood to be in particular a time period or time window during which MR TX signals are transmitted, while a non-transmit phase is understood to be in particular a time period or a time window during which no MR TX signals are transmitted. It is advantageous here that any interference in the reference noise signals from the noise source during a transmit phase is low to negligible.

Non-transmit phases can be, for example, time periods or phases between transmissions of excitation pulses of an MR sequence including a plurality of excitation pulses (spin echo train). Alternatively or additionally, non-transmit phases can be time periods or phases before or after MR sequences, which is particularly advantageous because no MR response signals of the patient occur then.

According to an embodiment, the Dicke switch is switched over during transmit phases of the MR system, and is connected alternately to the antenna or the noise source during non-transmit phases of the MR system. Any interference in the reference noise signal due to MR transmit signals is thereby prevented particularly effectively.

According to a development, the radiometer is only operated during non-transmit phases of the MR system, e.g. is only switched over during non-transmit phases of the MR system, and measured signals and reference signals that have been picked up are only evaluated during non-transmit phases of the MR system. Thereby likewise, any interference in the measurement of the thermal noise of the patient due to MR signals is reliably prevented. In addition, this development is particularly easy to implement since it is not necessary to allow for synchronization of a switchover of the Dicke switch during MR-transmit phases.

According to an embodiment, a useful band (i.e. the frequency band which is used to measure the thermal noise) of the radiometer lies outside an MR band (i.e. the frequency band used for MR signals). This has the advantage that any interference in the radiometric temperature measurement due to MR signals is particularly low. In other words, in order to avoid interference in the noise signals due to MR signals (MR transmit (TX) and possibly also MR receive (RX) signals), the useful band of the radiometer lies outside the MR frequency band. This can be realized, e.g., by the radiometer antenna having a useful band that lies outside the MR band. The MR band includes, in particular, a precession frequency of protons and lies at 42.5 MHz/T, for example. In the case of an MR device having a Bo field with a strength of 7 Tesla, the MR band extends, e.g., as far as approximately 300 MHz.

According to an advantageous development for achieving a high bandwidth, the useful band of the radiometer lies above the MR band, i.e., at higher frequencies. According to a particularly advantageous development for effective signal separation, a frequency interval between the MR band and the useful band of the radiometer is at least 5 MHz, in particular at least 10 MHz. However, larger frequency intervals are even more suitable for signal separation. A frequency interval of at least 50 MHz, specifically at least 100 MHz, is particularly suitable. Therefore in the case of a 1.5-T MR scanner, a lower limit of the useful band of the radiometer antenna is advantageously at least approximately 70 MHz, in particular at least approximately 150 MHz, and in the case of a 7-T MR scanner, it is at least approximately 303 MHz, in particular at least approximately 400 MHz, etc.

An upper limit of the useful band of the radiometer is essentially unlimited and can be, e.g., no more than 60 GHz, in particular no more than 10 GHz. When configuring the useful band, it can be taken into consideration in this case that a measuring depth of the radiometer decreases as the measuring frequency increases, and therefore if a desired measurement of the body temperature is particularly deep in the patient, the lowest possible useful frequencies should be selected. It has been shown specifically that a frequency interval in the range between 50 MHz and 150 MHz results in a good compromise between preventing cross-talk and measuring the temperature at sufficient depth in the patient.

According to an embodiment, the radiometer has a low-noise amplifier and at least one measured signal, which is measured by the radiometer antenna and then amplified by the amplifier, or noise signal is supplied to an evaluation device and evaluated by the evaluation device, e.g., in order to determine the body temperature and/or compare this with corresponding limit temperatures. On the basis of the evaluation, the signal strength of the MR transmit signal can be adapted automatically or according to user input. The noise signal that is picked up and amplified can be transferred to the evaluation device in analog or digital format.

The evaluation device can be a different device from the control device, but coupled thereto. According to a development, the evaluation device is integrated in the MR body coil, in which case in particular only an evaluated signal (digital or analog) is transferred from the MR body coil to the control device that is external thereto. According to a further development, the evaluation device is arranged externally to the MR body coil. The evaluation therefore takes place outside the MR body coil, whereby the MR body coil can advantageously be embodied in a particularly simple and economical manner. According to a development, the evaluation device can be integrated in the control device.

According to a development, the noise signal supplied to the evaluation device has its original frequency. This development allows the provision of a particularly simple and economical MR body coil.

According to an embodiment, the noise signal that is supplied to the evaluation device has a converted frequency (carrier frequency). In this case, the amplified noise signal is frequency converted while still in the MR body coil and is transferred to the outside in a different frequency band, in particular to the control transfer. In particular, if the noise signal is down-converted before transmission thereof, the advantage is achieved that cable attenuation can be kept low and any components of the body coil that are already present and already used for, e.g., signal transfer of MR signals can be shared. The down-conversion can be performed using any desired known methods, e.g., modulation of the measured signal onto a carrier wave of lower frequency.

According to an embodiment, the measured temperature or temperature distribution is displayed at a user interface of the MR system, e.g., at a user workstation. This allows monitoring of the body temperature by an operator, which can further increase safety for a patient. The temperature or temperature distribution can be used in particular to provide a parameter to the operator for the purpose of monitoring the state and/or well-being of the patient (in the same way as, e.g., a pulse, respiration or oxygen saturation) or to identify how the body (possibly weakened by illness) is reacting to the SAR load of the MR examination.

The object is also achieved by an MR body coil for use in the method described above. The MR body coil can be designed in a similar manner to the method and offers the same advantages.

In addition to the usual components for MR measurement, e.g., at least one MR antenna etc., the MR body coil has at least one radiometer antenna and an amplifier for amplifying the measured signals received by the radiometer antenna. The amplifier is a low-noise amplifier, in particular. Such an MR body coil can be embodied in a particularly economical manner. The at least one radiometer antenna advantageously differs from the at least one MR antenna.

According to an embodiment, the MR body coil also has a noise source and a Dicke switch of a Dicke radiometer. Signal paths of the noise signals can advantageously be kept short thereby.

Alternatively, the noise source and the Dicke switch can be arranged outside the MR body coil, e.g., in an evaluation device. This has the advantage that the MR body coil can be embodied in a particularly simple and economical manner and furthermore the reference noise signal generated by the noise source does not suffer interference from the MR signals.

According to a development, the MR body coil includes at least part of the evaluation device. Signal paths of the noise signals can advantageously be kept particularly short thereby.

According to an embodiment, the MR body coil has an input blocking filter between the radiometer antenna and the amplifier. Said blocking filter is configured to block the MR band. The input blocking filter allows MR signals to be prevented from interfering with the measured signals that are picked up by the radiometer antenna in a particularly reliable manner, since they are blocked. It is also thereby possible to ensure in a particularly reliable manner that the radiometer does not overmodulate during MR transmit phases. According to a development, the input blocking filter is connected downstream of the Dicke switch, since in this way the reference noise signal generated by the noise source also undergoes the same processing by the input blocking filter as the measured signal and therefore also experiences any identical power loss that might occur. The accuracy of the radiometric temperature measurement is again improved thus.

According to a development, the radiometer antenna is arranged within a housing of the MR body coil. According to a development, the housing is impedance-matched in the region between the radiometer antenna and the anticipated position of the patient. It is thereby possible advantageously to prevent standing wave effects between patient and radiometer antenna. The impedance matching can be achieved, e.g., by corresponding adaptation of a thickness of the housing in this region and/or by selecting a dielectric constant of the housing wall in this region. In the event that the MR body coil is intended to be arranged underneath a patient (e.g. a spine coil, head coil or head/neck coil), a cushion on which the patient is intended to lie can alternatively or additionally be embodied for impedance matching, e.g., by its choice of material.

According to a development, the MR body coil is a head coil, a head/neck coil, a spine coil and/or an abdomen coil.

The object is further achieved by an MR system that is designed to perform the method as described above and has at least one MR body coil as described above and a control device that is connected to the at least one MR body coil. The MR system can be designed in a similar manner to the method and offers the same advantages. According to a development, the MR system further includes an MR scanner that can be controlled by the control device in particular.

### BRIEF DESCRIPTION OF THE DRAWINGS

The properties, features and advantages of this invention as described above and the manner in which these are achieved become clearer and easier to understand in the context of the following schematic description of exemplary embodiments which are explained in greater detail with reference to the drawings. For the sake of clarity, identical or functionally identical elements may be denoted by the same reference signs in this case.

FIG. 1 shows an MR system with a radiometer antenna according to one embodiment.

### DETAILED DESCRIPTION OF THE PREFERRED EMBODIMENTS

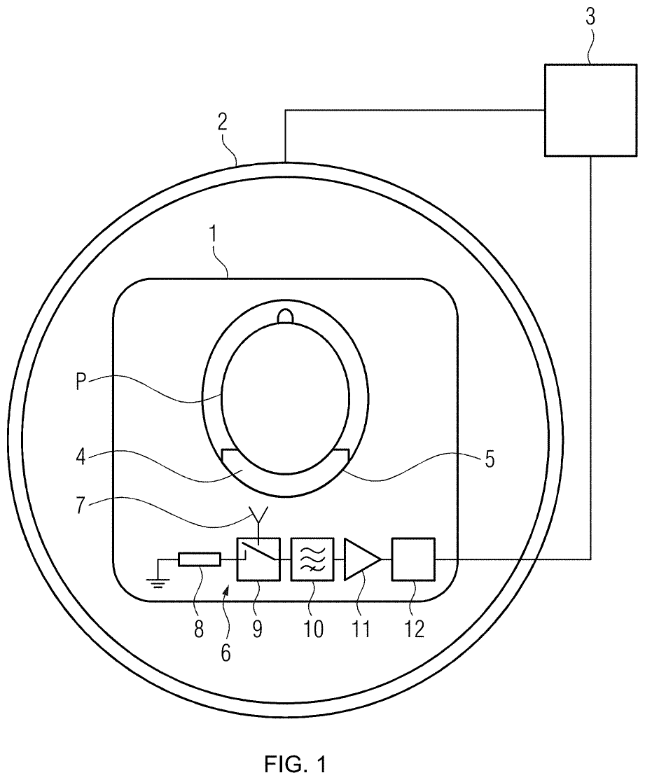

FIG. 1 shows, in a sectional view from the head side, an outline of an MR system including an MR body coil 1, an MR scanner 2 and a control device 3 connected to the MR scanner 2 and the MR body coil 1. The MR scanner 2 is in particular a high-field scanner whose Bo field strength is at least 1.5 T, e.g. 1.5 T, 3 T, 7 T, 10 T, etc. The MR frequency band of the MR scanner 2 lies in the region of 42.4 MHz/T. As usual, the MR scanner 2 is equipped with MR transmit antennas (not shown) in order to transmit MR pulses at frequencies in the MR band during an MR scan, e.g., in the context of echo trains. The MR response signals of a patient P are picked up by MR receive antennas (not shown) of the MR body coil 1. The activation of the MR scanner 2, the evaluation of the signals picked up by the MR receive antennas, and imaging are performed by the control device 3 in a manner which is generally known.

The MR body coil 1 is designed here by way of example as a head coil for examining a head region of the patient P. The head of the patient P rests on top of a cushion 4 on a housing wall 5 of the MR body coil 1.

The MR body coil 1 contains at least some components of a Dicke radiometer 6, namely here a radiometer antenna 7, a noise source 8 in the form of a grounded 50-Ohm resistance, a Dicke switch 9 that can be switched over between the radiometer antenna 7 and the noise source 8 with a specified switchover frequency, an input blocking filter (e.g. low-pass filter) 10 that is connected downstream of the Dicke switch 9 for the purpose of blocking frequencies of the MR band, and connected downstream of the input blocking filter 10 is a low-noise amplifier 11 and possibly further electronic components 12 such as a frequency converter for down-conversion of the incoming signals, with e.g. local oscillator, mixer, IF filter, A/D converter, microprocessor, etc.

The radiometer antenna 7 and the low-noise amplifier 11 both work in a useful band which lies above the MR band with a frequency interval. In the case of a 7-T MR scanner, the start of the useful band is e.g. at least 303 MHz, advantageously approximately 400 MHz, and can extend as far as e.g. 10 GHz or even higher, e.g. up to 60 GHz. The input blocking filter 10 can block frequencies below the useful band accordingly.

The generated noise signals can be down-converted by a frequency converter 12 before transfer to the control device 3 in order to keep cable attenuation low, for example, and to be able to share the use of any existing components of the MR body coil 1 that are already used for, e.g., signal transfer of MR signals.

The control device 3 is configured to determine from the noise signals of the Dicke radiometer 6 a body temperature of the patient P in the field of view or illumination region of the radiometer antenna 7. The body temperature advantageously is or includes an internal body temperature of the patient P that cannot be obtained using, e.g., IR cameras.

The control device 3 is also configured, e.g., programmed, to compare the measured body temperature with a specified temperature limit value and to adapt the MR transmit power of the MR scanner 2 accordingly.

In order to reduce any impedance mismatch between the patient P and the radiometer antenna 7, the cushion 4 and the housing wall 5 are impedance-matched.

The MR body coil 1 can include one or a plurality of Dicke radiometers 6.

Although the invention is illustrated and described in detail by the exemplary embodiments shown herein, the invention in not limited to these, and other variations can be derived therefrom by a person skilled in the art without thereby departing from the scope of the invention.

For example, one or a plurality of the components of the Dicke radiometer 6 shown in FIG. 1 as belonging to the MR body coil 1 can alternatively be present in the control device 3, e.g., the noise source 8 (which can also take the form of a noise diode), the Dicke switch 9, the input blocking filter 10, and/or further electronic components 12.

In general, “one”, “a”, etc. can be understood to signify single or multiple instances, particularly in the sense of “at least one” or “one or more”, etc., unless explicitly stated otherwise, e.g. by the expression “precisely one”, etc.

Likewise, a numerical specification can encompass both the number specified and a normal tolerance range unless explicitly stated otherwise.

1. A method for operating an MR system comprising at least one MR body coil and a control device connected to the at least one MR body coil, the method comprising:
measuring, by at least one radiometer, at least one body temperature of a body region, which body region can be illuminated by the respective radiometer, of a patient to be examined by the MR system,
comparing the measured body temperature with a limit temperature, and
bringing an MR transmit power directed at the patient closer to the limit temperature on the basis of a result of the comparison.

2. The method as claimed in claim 1, wherein the radiometer is a Dicke radiometer comprising a radiometer antenna, a noise source and a Dicke switch, and the Dicke switch is switched over alternately between the radiometer antenna and the noise source.

3. The method as claimed in claim 2, wherein the Dicke switch is connected to the noise source during transmit phases of the MR system and is connected to the radiometer antenna during non-transmit phases of the MR system.

4. The method as claimed in claim 2, wherein the Dicke switch is switched over during transmit phases of the MR system, such that the Dicke switch is connected alternately to the radiometer antenna and the noise source during non-transmit phases of the MR system.

5. The method as claimed in claim 1, wherein a useful band of the radiometer lies outside an MR band.

6. The method as claimed in claim 1, wherein the radiometer has a low-noise amplifier and at least one measured signal measured by the radiometer antenna and then amplified by the amplifier is supplied to and is evaluated by the control device.

7. The method as claimed in claim 6, wherein the noise signal supplied to the control device has its original frequency.

8. The method as claimed in claim 6, wherein the noise signal supplied to the control device has a down-converted frequency.

9. The method as claimed in claim 1, wherein the measured body temperature is displayed at a user interface of the MR system.

10. The method as claimed in claim 1, wherein measuring by the radiometer comprises measuring by a plurality of radiometers for respectively different body regions that can be illuminated a plurality of body temperatures at different depths of the patient to be examined.

11. The method of claim 5, wherein the useful band is above the MR band with a frequency interval of at least 5 MHz.

12. The method of claim 2, wherein the radiometer has a low-noise amplifier and at least one measured signal measured by the radiometer antenna and then amplified by the amplifier is supplied to and is evaluated by the control device.

13. The method as claimed in claim 6, wherein measuring by the radiometer comprises measuring by a plurality of radiometers for respectively different body regions that can be illuminated a plurality of body temperatures at different depths of the patient to be examined.

14. An MR body coil comprising:
an MR antenna;
at least one radiometer antenna, and
an amplifier connected downstream of the radiometer antenna.

15. The MR body coil as claimed in claim 14, further comprising an input blocking filter connected downstream of the radiometer antenna and is designed to block the MR band.

16. The MR body coil as claimed in claim 14, wherein a housing wall of the MR body coil and/or a cushion for supporting the patient, situated between the radiometer antenna and the patient is impedance-matched.

17. The MR body coil as claimed in claim 14, wherein the MR body coil is a head coil, a head/neck coil, a spine coil and/or an abdomen coil.

18. An MR system comprising:
at least one MR body coil comprising at least one radiometer antenna, and an amplifier connected downstream of the radiometer antenna;
a control device (connected to the at least one MR body coil, the control device configured to compare a measured body temperature from the amplifier with a limit temperature, and bring an MR transmit power closer to the limit temperature on the basis of a result of the comparison.
